(19) United States
(12) Patent Application Publication
(10) Pub. No.: US 2024/0008884 A1
(43) Pub. Date: Jan. 11, 2024

(54) FIRST ORDER PERCUTANEOUS MECHANICAL ARTERIAL ADVANCED THROMBECTOMY TECHNOLOGY

(71) Applicant: Duangnapa Somsuppaphon Cuddy, Bloomington, IN (US)

(72) Inventor: Duangnapa Somsuppaphon Cuddy, Bloomington, IN (US)

(21) Appl. No.: 18/219,998

(22) Filed: Jul. 10, 2023

Related U.S. Application Data
(60) Provisional application No. 63/359,816, filed on Jul. 9, 2022.

(51) Int. Cl. A61B 17/22; A61B 17/221

(52) U.S. Cl. CPC A61B 17/22; A61B 17/221; A61B 17/2202; A61B 2017/22082; A61B 2017/22067; A61B 2017/22079; A61B 2017/00893

(57) ABSTRACT

A method for thrombus removal within a branched artery includes accessing the branched artery percutaneously; placing a balloon occlusion at a contralateral branch of the branched artery; placing a funneled sheath at the ipsilateral branch of the branched artery; and removing the thrombus mechanically.

### CROSS REFERENCE TO RELATED APPLICATIONS

This application claims priority to U.S. Provision Application No. 63/359,816, filed on Jul. 9, 2022, hereby incorporated by reference in its entirety.

### FIELD OF THE INVENTION

The present disclosure relates to methods for the mechanical removal of thrombus from arteries, and particularly to methods for the percutaneous mechanical remove of thrombus from an aorta-iliac-first order artery.

### BACKGROUND

Endovascular aneurysm repair (EVAR) is often the primary choice for treating abdominal aortic aneurysms (AAAs) and iliac artery aneurysms. EVAR is oftentimes successful and has a lower mortality rate compared to open surgery for AAA repairs. Despite that benefit, EVAR has a higher re-intervention rate, meaning patients that undergo EVAR have a higher chance of requiring additional follow-up procedures. Some studies show a re-intervention rate as high as 20%.

Limb occlusion, the thrombotic obstruction of blood flow in one or both endograft limbs, is a major cause of re-hospitalization following EVAR. Generally endograft limb occlusion following EVAR is treated with open surgery allowing thrombectomy and/or bypass. Risks of operative femoral cut-down include nerve damage and a 2.6% risk of wound complications with associated extended length of stay. Current knowledge of endovascular approaches to this complication includes case reports with thrombolytic use and expense which also note AKI, blood loss, and hematuria.

Limb occlusion can be symptomatic or asymptomatic. Symptomatic aorto-iliac artery thrombosis causes significant morbidity and mortality, which often requires major vascular surgery, which may also lead to more additional complications and a prolonged hospital stay.

Accordingly, it would be desirable to provide a method for an endovascular approach to limb occlusion repair following EVAR that alleviated or addressed one or more of the above-discussed concerns associated with conventional methods, or alleviated or addressed one or more other concerns or disadvantages, or provided one or more advantages by comparison with prior methods.

### BRIEF SUMMARY

A method for thrombus removal within a branched artery is disclosed herein.

In accordance with embodiments of the present disclosure, a method for thrombus removal within a branched artery comprises accessing the branched artery percutaneously; placing a balloon occlusion at a contralateral branch of the branched artery; placing a funneled sheath at the ipsilateral branch of the branched artery; and removing the thrombus mechanically.

In embodiments, the branched artery is an iliac artery, or an aorta-iliac first order branch artery.

In accordance with some embodiment, the method further comprises the step of identifying and locating a thrombus in the branched artery. In some embodiments, the step of identifying and locating the thrombus comprises using a method selected from the group consisting of symptom analysis, computed tomography angiography (CTA), and combinations thereof.

In some embodiments, the step of accessing the branched artery percutaneously comprises using ultrasound. In accordance with some embodiments, wherein the branched artery is an aorta-iliac-first order branch artery, and the step of accessing the branched artery percutaneously is through bilateral common femoral arteries.

In accordance with embodiments of the present disclosure, the method further includes placing a sheath at both the contralateral branch and ipsilateral branch. In some embodiments, the sheath is selected from the group consisting of 5F sheaths, 7F sheaths, and combinations thereof. In some embodiments, the funneled sheath is a 16F sheath.

In some embodiments, the step of removing the thrombus mechanical comprises percutaneous transluminal catheter aspiration.

In accordance with some embodiments, the method further comprising administering heparin. In some embodiments, the method is free from any step of or including administering alteplase.

A method for thrombus removal within an aorta-iliac-first order artery is disclosed herein.

In accordance with embodiments of the present disclosure, a method for thrombus removal within an aorta-iliac-first order artery comprises accessing the aorta-iliac-first order artery percutaneously via bilateral common femoral arteries; placing a balloon occlusion at a first iliac artery; placing a funneled sheath at a second iliac artery, wherein the thrombus is located in the second iliac artery; and removing the thrombus mechanically.

In some embodiments, prior to placing a balloon occlusion at a first iliac artery, a first sheath is placed at the first iliac artery and a second sheath is placed at the second iliac artery.

In some embodiments, the step of removing the thrombus mechanically comprises percutaneous transluminal catheter aspiration. In accordance with some embodiments, the funneled sheath is a sheath containing a nitinol funnel extending from a distal end of the sheath. In some embodiments, the step of removing the thrombus mechanically further comprises using a ClotTriever sheath at the second iliac artery.

In some embodiments, the method is free from administering alteplase. In some embodiments, the method further includes administering heparin.

### BRIEF DESCRIPTION OF THE DRAWINGS

Embodiments of the method and related methods of operation are disclosed with reference to the accompanying drawings and are for illustrative purposes only. The method encompassed herein are not limited in their applications to the details of construction, arrangements of components, or other aspects or features illustrated in the drawings, but rather such apparatuses and methods encompassed herein include other embodiments or are capable of being practiced or carried out in other various ways. Like reference numerals are used to indicate like components. In the drawings:

FIG. 1 is a schematic illustrating a method of thrombus removal; and

FIG. 2 is a representative angiogram after thrombus removal and revascularization.

### DETAILED DESCRIPTION

In embodiments of the present disclosure, a method for thrombus removal within a branched artery.

In an embodiment, the method includes identifying and locating an arterial thrombus. In an embodiment, the step of identifying and locating the arterial thrombus includes using one or more of symptom analysis, computed tomography angiography (CTA), and other methods of finding and identifying an arterial thrombus.

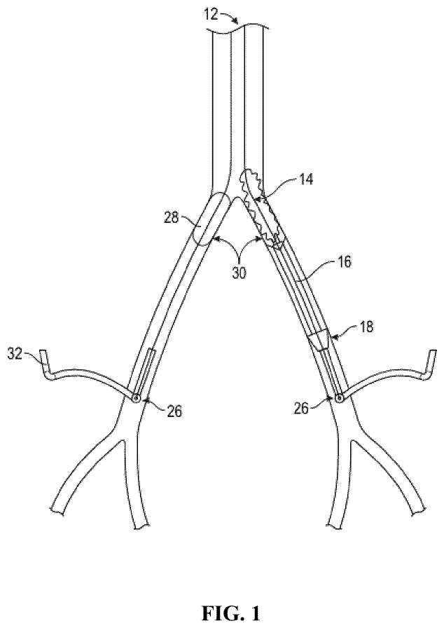

In a particular embodiment, and as shown in FIG. 1, the arterial thrombus 14 is more specifically located in the aorta 12, at the iliac arteries 30, and more specifically within an aorta-iliac-first order branch artery.

The method for thrombus removal within an artery, and particularly an aorta-iliac-first order branch artery, includes accessing the artery percutaneously, that is, without surgical incision. Percutaneously access may be assisted by ultrasound. In a particular embodiment, when the thrombus is located in an aorta-iliac-first order branch artery, access is obtained via the bilateral common femoral arteries (CFAs) 26, as shown in FIG. 1.

The method further includes placing a balloon occlusion 28 on the opposite side (contralateral) as the thrombus, as shown in FIG. 1. The use of a balloon occlusion 28 removes the possibility of an embolizing clot from the contralateral side forming and moving to the ipsilateral side.

In an embodiment, the method further includes deploying a suture device using the preclose technique. In a preferred embodiment, the step of deploying a suture device using the preclose technique occurs prior to any subsequent steps in the method. In embodiments in which the preclose technique is used, the balloon occlusion 28 is placed prior to the step of deploying a suture device using the preclose technique.

In an embodiment, a sheath 32 may be placed to the bilateral arteries to establish proper access. In a particular embodiment, the sheaths are 5F sheaths or 7F sheaths. In some embodiments, more than one sheath may be used. In a particular embodiment, first sheaths are placed bilaterally, and a second sheath is placed on the contralateral side (side opposite the thrombus). The sheaths may be placed using angiographic guidance.

In embodiments, the sheath 32 may be placed before, simultaneously with, or after the deployment of the balloon occlusion 28. In still further embodiments, when the preclose technique is used, the sheath 32 may be placed before, simultaneously with, or after the deployment of the suture device using the preclose technique.

The method further includes mechanical removal of the thrombus. In a particular embodiment, the step of mechanical removal of the thrombus comprises placing a funneled sheath 18 on the same side (ipsilateral) as the thrombus, as shown in FIG. 1. In a particular embodiment, the funneled sheath 18 is a 16F sheath.

In some embodiments, the step of mechanical removal of the thrombus includes percutaneous transluminal catheter aspiration. In an embodiment, the percutaneous transluminal catheter aspiration uses a 16F thrombectomy catheter 16 through a 16F Inari FlowTriever catheter (Inari Medical), with a ClotTreiver sheath at the contralateral CFA 26.

In an embodiment, the method includes administering heparin. In an embodiment, the heparin is administered intravenously during the duration of the method.

In an embodiment, the method is wholly free from any step of or including administering alteplase (tPA).

In an embodiment, a blood return system is used to return blood to contralateral arterial access.

### Experimental Data

A 64-year old man with hyperlipidemia, chronic obstructive pulmonary disease, and a 40-year history of smoking was found incidentally to have a 5.6 cm abdominal aortic aneurysm (AAA). He underwent percutaneous EVAR with the use of Medtronic Endurant IIs (28×14×103-mm main body and 16×13×124-mm limbs). Intravascular ultrasound (IVUS) demonstrated adequate expansion of all stent graft components, and completion angiography demonstrated a patent endograft and no endoleaks. The patient returned home the next day.

The patient presented to the emergency room 3 months later with a 1-week history of left leg claudication and episodic extremity coolness and numbness. Despite counseling for smoking cessation, the patient and continued to smoke regularly and was noncompliant with antiplatelet therapy. His physical examination revealed decreased left lower extremity pulses and a sensory deficit. Computed tomography angiography (CTA) demonstrated acute thrombosis of the left iliac limb of the stent graft with preserved vasculature distally.

Because of this thrombosis and the patient's symptoms, the preoperative plan was endovascular mechanical limb thrombectomy with possible graft revision.

Vascular and wire access was obtained via the bilateral common femoral arteries (CFAs) using ultrasound guidance. Next, 5F sheaths were placed to the bilateral CFAs with angiographic guidance to confirm adequate access. A 7F sheath was placed over the wire to the right CFA. The patient received intravenous heparin for systemic anticoagulation throughout the operation. Before preclosure device delivery at the left CFA, an occlusion balloon was placed over the wire to the proximal right limb. This balloon was deployed before delivery of the preclose devices and maintained during thrombectomy to remove the possibility of an embolizing clot from the left limb to the right iliac system. A preclosure technique was used in the left CFA, followed by dilation and delivery of a 16F ClotTriever sheath (Inari Medical, Irvine, CA).

Percutaneous transluminal catheter aspiration allowed for manual thrombectomy of the left stent graft limb via a 16F thrombectomy catheter through a 16F Inari FlowTriever catheter (Inari Medical) with a ClotTriever sheath at the left CFA, and a FlowSaver blood return system was used to return blood to the contralateral arterial access. This sheath was significant because it has a nitinol funnel extending from the distal extent, enabling simultaneous blood flow, thrombus capture, and distal embolization mitigation. Subsequently, transluminal thromboembolectomy using a 12×40-mm balloon was performed.

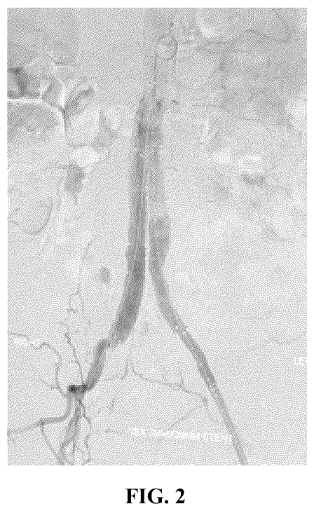

The complication of limb occlusion was confirmed by IVUS and angiography, enabling left limb treatment: (1) competitive flow due to contralateral limb deployment or proximal migration of 3 to 5 mm; and (2) underexpansion due to external compression from a common iliac artery requiring balloon-expandable stent placement and hypogastric coverage. Revision of the left stent graft limb was accomplished by placement of a 16×10×124-mm stent graft proximally, followed by deployment of a 7×39-mm balloon expandable stent to distal left graft limb. After angioplasty, the repair was visualized via IVUS, which demonstrated adequate stent graft expansion. Completion angiography revealed no evidence of thrombus within the sheath's funnel. The blood loss was 200 mL, and no thrombolytic agent was administered. A representative completion angiogram after revascularization is shown in FIG. 2.

The patient returned home the following day without hematuria or acute kidney injury (AKI). At the 3-month outpatient follow-up, he was free of complaints and had not experienced access site complications. CTA at the time revealed no endoleak and a patent endograft. This postoperative CTA had also demonstrated no injury, dissection, or stenosis as a result of the funnel sheath.

From the foregoing, it will be appreciated that although specific examples have been described herein for purposes of illustration, various modifications may be made without deviating from the spirit or scope of this disclosure. It is therefore intended that the foregoing detailed description be regarded as illustrative rather than limiting, and that it be understood that it is the following claims, including all equivalents, that are intended to particularly point out and distinctly claim the claimed subject matter.

It is specifically intended that the present invention not be limited to the embodiments and illustrations contained herein, but include modified forms of those embodiments including portions of the embodiments and combinations of elements of different embodiments as come within the scope of the following claims.

What is claimed is:

1. A method for thrombus removal within a branched artery, the method comprising:
accessing the branched artery percutaneously;
placing a balloon occlusion at a contralateral branch of the branched artery;
placing a funneled sheath at the ipsilateral branch of the branched artery; and
removing the thrombus mechanically.

2. The method of claim 1, wherein the branched artery is an iliac artery.

3. The method of claim 2, wherein the iliac artery is an aorta-iliac first order branch artery.

4. The method of claim 1, further comprising the step of identifying and locating a thrombus in the branched artery.

5. The method of claim 2, wherein the step of identifying and locating the thrombus comprises using a method selected from the group consisting of symptom analysis, computed tomography angiography (CTA), and combinations thereof.

6. The method of claim 1, wherein the step of accessing the branched artery percutaneously comprises using ultrasound.

7. The method of claim 1, wherein the branched artery is an aorta-iliac-first order branch artery, and accessing the branched artery percutaneously is through bilateral common femoral arteries.

8. The method of claim 7, wherein the method further includes placing a sheath at both the contralateral branch and ipsilateral branch.

9. The method of claim 8, wherein the sheath is selected from the group consisting of 5F sheaths, 7F sheaths, and combinations thereof.

10. The method of claim 1, wherein the funneled sheath is a 16F sheath.

11. The method of claim 1, wherein the step of removing the thrombus mechanical comprises percutaneous transluminal catheter aspiration.

12. The method of claim 1, further comprising administering heparin.

13. The method of claim 1, wherein the method is free from any step of or including administering alteplase.

14. A method for thrombus removal within an aorta-iliac-first order artery, the method comprising:
accessing the aorta-iliac-first order artery percutaneously via bilateral common femoral arteries;
placing a balloon occlusion at a first iliac artery;
placing, after the placing of the balloon occlusion at the first iliac artery, a funneled sheath at a second iliac artery, wherein the thrombus is located in the second iliac artery; and
removing the thrombus mechanically.

15. The method of claim 14, wherein, prior to placing a balloon occlusion at a first iliac artery, a first sheath is placed at the first iliac artery and a second sheath is placed at the second iliac artery.

16. The method of claim 14, wherein the step of removing the thrombus mechanically comprises percutaneous transluminal catheter aspiration.

17. The method of claim 16, wherein the funneled sheath is a sheath containing a nitinol funnel extending from a distal end of the sheath.

18. The method of claim 17, wherein the step of removing the thrombus mechanically further comprises using a ClotTriever sheath at the second iliac artery.

19. The method of claim 14, wherein the method is free from administering alteplase.

20. The method of claim 14, further including the step of administering heparin.
